(19) United States
(12) Patent Application Publication
(10) Pub. No.: US 2026/0308523 A1
(43) Pub. Date: Oct. 8, 2026

(54) COMPOSITION FOR INDUCING DENDRITIC CELL DIFFERENTIATION, COMPRISING METAL-ORGANIC FRAMEWORK, AND USE THEREOF

(71) Applicant: MEDIARK INC., Cheongju-si, Chungcheongbuk-do (KR)

(72) Inventors: Se Na KIM, Sejong (KR); Nea Young CHUN, Suwonsi, Gyeonggi-do (KR); Yu Jin OH, Sejong (KR)

(21) Appl. No.: 19/481,569

(22) PCT Filed: Oct. 24, 2025
(86) PCT No.: PCT/KR2024/016228

§ 371 Date: Nov. 5, 2025

(30) Foreign Application Priority Data
Dec. 29, 2023 (KR) 10-2023-0196985

(51) Int. Cl. C12N 5/0784; C07F 5/06; C07F 7/00; C07F 15/02

(52) U.S. Cl. CPC C12N 5/0639; C07F 5/06; C07F 7/003; C07F 15/025; C12N 2501/22; C12N 2501/999; C12N 2506/1353

(57) ABSTRACT

An embodiment relates to a composition for inducing dendritic cell differentiation, comprising a metal-organic framework, and a use thereof, the composition having the effect of not only enabling the increasing of the expression of surface protein markers of dendritic cells, but also enabling the inducing of differentiation into dendritic cells having enhanced viability and migration properties when administered into an individual.

### TECHNICAL FIELD

The present invention relates to a composition for inducing differentiation of dendritic cells including a metal-organic framework and the use thereof.

### BACKGROUND ART

Dendritic cells (DCs) are immune cells that form part of the mammalian immune system and act as professional antigen-presenting cells (APCs) that process antigen material and present it on the surface to other cells of the immune system.

Dendritic cells are present in small quantities in tissues that are in contact with the external environment, mainly the skin and the inner lining of the nose, lungs, stomach and intestines. They are composed of a heterogeneous population with a phenotype distinct from that of macrophages.

Specifically, dendritic cells are the only immune cells that have the ability to induce a primary immune response, which can stimulate naive T cells that have never been exposed to an antigen, and also have the ability to induce immune memory. It is known that dendritic cells can function to activate a strong immune response as described above because they act as antigen-presenting cells (APCs) that display high concentrations of not only MHC molecules (I/II) on the cell surface, but also costimulatory molecules such as CD80 and CD86, and adhesion molecules such as ICAM-1, and secrete various cytokines (IFN-alpha, IL-12, IL-18, etc.).

As described above, dendritic cells are the most powerful antigen-presenting cells. Although they are present in extremely small numbers in the body, they have an excellent function of regulating T cell immunity, and thus have been actively studied as a therapeutic agent for cancer or infectious diseases in clinical studies to induce immunity against specific antigens.

Specifically, it has been found that, when immature dendritic cells isolated from tissue or blood are stimulated with an antigen in vitro, to differentiate into mature dendritic cells which are then injected in vivo, they exhibit immunogenicity. If this technology is utilized, the dendritic cells may have great application value as a cell vaccine for inducing immunity against specific antigens of cancer or pathogens.

Accordingly, with regard to the technology for differentiating immature dendritic cells isolated from tissue or blood into mature dendritic cells, there is a need for the development of a technology for improving the differentiation potential.

### PRIOR ART DOCUMENTS

### Patent Documents

KR 10-2013-0023797 A

### DISCLOSURE

### Technical Problem

An object of the present invention is to provide a composition for inducing differentiation of dendritic cells including a metal-organic framework and the use thereof.

Another object of the present invention is to provide a composition for inducing differentiation of dendritic cells including a metal-organic framework that may not only increase the expression of dendritic cell surface protein markers but also induce differentiation into dendritic cells with increased viability and migratory ability when administered into a subject.

Still another object of the present invention is to provide a method for inducing differentiation of dendritic cells using the composition.

### Technical Solution

In order to achieve the above objects, a composition for inducing differentiation of dendritic cells according to one embodiment of the present invention includes a metal-organic framework (MOF).

The metal-organic framework includes: a metal cluster; and a ligand compound represented by Formula 1 below that coordinates to the metal cluster:

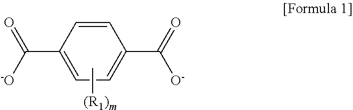

wherein
m is an integer ranging from 0 to 4, and
R₁ is selected from the group consisting of hydrogen, deuterium, a cyano group, a nitro group, an amino group, a halogen group, a hydroxy group, a substituted or unsubstituted alkylthio group having 1 to 4 carbon atoms, a substituted or unsubstituted alkyl group having 1 to 30 carbon atoms, a substituted or unsubstituted cycloalkyl group having 3 to 20 carbon atoms, a substituted or unsubstituted alkenyl group having 2 to 30 carbon atoms, a substituted or unsubstituted alkynyl group having 2 to 24 carbon atoms, a substituted or unsubstituted aralkyl group having 7 to 30 carbon atoms, a substituted or unsubstituted aryl group having 6 to 30 carbon atoms, a substituted or unsubstituted heteroaryl group having 1 to 60 carbon atoms, a substituted or unsubstituted heteroarylalkyl group having 2 to 30 carbon atoms, a substituted or unsubstituted alkoxy group having 1 to 30 carbon atoms, a substituted or unsubstituted alkylamino group having 1 to 30 carbon atoms, a substituted or unsubstituted arylamino group having 6 to 30 carbon atoms, a substituted or unsubstituted aralkylamino group having 7 to 30 carbon atoms, a substituted or unsubstituted heteroarylamino group having 1 to 24 carbon atoms, a substituted or unsubstituted alkylsilyl group having 1 to 30 carbon atoms, a substituted or unsubstituted arylsilyl group having 6 to 30 carbon atoms, and a substituted or unsubstituted aryloxy group having 6 to 30 carbon atoms.
R₁ is selected from the group consisting of an amino group, a substituted or unsubstituted alkylamino group having 1 to 30 carbon atoms, a substituted or unsubstituted arylamino group having 6 to 30 carbon atoms, a substituted or unsubstituted aralkylamino group having 7 to 30 carbon atoms, and a substituted or unsubstituted heteroarylamino group having 1 to 24 carbon atoms.

The metal-organic framework includes at least one metal or metal ion selected from the group consisting of aluminum (Al), iron (Fe), and zirconium (Zr).

A method for inducing differentiation of dendritic cells according to another embodiment of the present invention includes a step of treating immature dendritic cells with the composition, followed by culturing.

The immature dendritic cells are cells obtained by treating bone marrow cells with granulocyte-macrophage colony-stimulating factor (GM-CSF).

A dendritic cell according to another embodiment of the present invention is a cell differentiated by the method for inducing differentiation.

The dendritic cell has increased expression of at least one surface protein marker selected from the group consisting of CD40, CD80, CD86, MHC class I, and MHC class II.

In the present invention, unless specifically stated otherwise, “hydrogen” is hydrogen, protium, deuterium, or tritium.

In the present invention, the “halogen group” is fluorine, chlorine, bromine, or iodine.

In the present invention, the “alkyl” means a monovalent substituent derived from a straight or branched chain saturated hydrocarbon having 1 to 40 carbon atoms. Examples thereof include, but are not limited to, methyl, ethyl, propyl, isobutyl, sec-butyl, pentyl, iso-amyl, hexyl, and the like.

In the present invention, the “alkenyl” means a monovalent substituent derived from a straight or branched chain unsaturated hydrocarbon having 2 to 40 carbon atoms and having at least one carbon-carbon double bond. Examples thereof include, but are not limited to, vinyl, allyl, isopropenyl, 2-butenyl, and the like.

In the present invention, the “alkynyl” means a monovalent substituent derived from a straight or branched chain unsaturated hydrocarbon having 2 to 40 carbon atoms and having at least one carbon-carbon triple bond. Examples thereof include, but are not limited to, ethynyl, 2-propynyl, and the like.

In the present invention, the “alkylthio” means the above-described alkyl group bonded through a sulfur linkage (—S—).

In the present invention, the “aryl” means a monovalent substituent derived from an aromatic hydrocarbon having 6 to 60 carbon atoms and having a single ring or a combination of two or more rings. In addition, it may also include a form in which two or more rings are simply attached (pendant) to each other or fused together, and specific examples thereof include, but are not limited to, a naphthyl group, an anthracenyl group, a phenanthryl group, a triphenyl group, a pyrenyl group, a phenalenyl group, a perylenyl group, a chrysenyl group, fluorenyl group, etc. The fluorenyl group may be substituted, and adjacent groups may be bonded to each other to form a ring.

In the present invention, the “heteroaryl” means a monovalent substituent derived from a monoheterocyclic or polyheterocyclic aromatic hydrocarbon having 6 to 30 carbon atoms. Here, at least one carbon in the ring, preferably 1 to 3 carbons, is/are substituted with a heteroatom such as N, O, S or Se. In addition, the heteroaryl may include a form in which two or more rings are simply attached (pendant) to each other or fused together. Furthermore, it may include a form fused with an aryl group. Examples of such heteroaryls include, but are not limited to, 6-membered monocyclic rings such as pyridyl, pyrazinyl, pyrimidinyl, pyridazinyl, and triazinyl; polycyclic rings such as phenoxathienyl, indolizinyl, indolyl, purinyl, quinolyl, benzothiazole, and carbazolyl; and 2-furanyl, N-imidazolyl, 2-isoxazolyl, 2-pyridinyl, 2-pyrimidinyl, etc.

In the present invention, the “aryloxy” means a monovalent substituent represented by RO—, wherein R is an aryl having 6 to 60 carbon atoms. Examples of the aryloxy include, but are not limited to, phenyloxy, naphthyloxy, diphenyloxy, and the like.

In the present invention, “alkyloxy” refers to a monovalent substituent represented by R′O—, wherein R′ represents an alkyl having 1 to 40 carbon atoms. The alkyloxy may include a linear, branched, or cyclic structure. Examples of the alkyloxy include, but are not limited to, methoxy, ethoxy, n-propoxy, 1-propoxy, t-butoxy, n-butoxy, pentoxy, and the like.

In the present invention, the “alkoxy” may be linear, branched or cyclic. The number of carbon atoms of the alkoxy is not particularly limited, but is preferably 1 to 20 carbon atoms. Specific examples of the alkoxy include, but are not limited to, methoxy, ethoxy, n-propoxy, isopropoxy, i-propyloxy, n-butoxy, isobutoxy, tert-butoxy, sec-butoxy, n-pentyloxy, neopentyloxy, isopentyloxy, n-hexyloxy, 3,3-dimethylbutyloxy, 2-ethylbutyloxy, n-octyloxy, n-nonyloxy, n-decyloxy, benzyloxy, p-methylbenzyloxy, and the like.

In the present invention, the “aralkyl” means an aryl-alkyl group, wherein the aryl and the alkyl are as defined above. Preferred aralkyls include lower alkyl groups. Non-limiting examples of suitable aralkyl groups include benzyl, 2-phenethyl, and naphthalenylmethyl. Bonding to the parent moiety is via the alkyl.

In the present invention, the “arylamino group” means an amine substituted with an aryl group having 6 to 30 carbon atoms.

In the present invention, the “alkylamino group” means an amine substituted with an alkyl group having 1 to 30 carbon atoms.

In the present invention, the “aralkylamino group” means an amine substituted with an aryl-alkyl group having 6 to 30 carbon atoms.

In the present invention, the “heteroarylamino group” means an amine group substituted with an aryl group having 6 to 30 carbon atoms and a heterocyclic group.

In the present invention, the “heteroaralkyl group” means an aryl-alkyl group substituted with a heterocyclic group.

In the present invention, the “cycloalkyl” means a monovalent substituent derived from a monocyclic or polycyclic non-aromatic hydrocarbon having 3 to 40 carbon atoms. Examples of the cycloalkyl include, but are not limited to, cyclopropyl, cyclobutyl, cyclopentyl, cyclohexyl, norbornyl, adamantine, and the like.

In the present invention, the “heterocycloalkyl” means a monovalent substituent derived from a non-aromatic hydrocarbon having 3 to 40 carbon atoms, wherein at least one carbon in the ring, preferably 1 to 3 carbons, is/are substituted with a heteroatom such as N, O, S or Se. Examples of the heterocycloalkyl include, but are not limited to, morpholine, piperazine, and the like.

In the present invention, “alkylsilyl” refers to silyl substituted with an alkyl having 1 to 40 carbon atoms, and “arylsilyl” refers to silyl substituted with an aryl having 6 to 60 carbon atoms.

In the present invention, the “fused ring” means a fused aliphatic ring, a fused aromatic ring, a fused heteroaliphatic ring, a fused heteroaromatic ring, or a combination thereof.

In the present invention, “adjacent groups may be bonded to each other to form a ring” means that adjacent groups are bonded to each other to form a substituted or unsubstituted aliphatic hydrocarbon ring; a substituted or unsubstituted aromatic hydrocarbon ring; a substituted or unsubstituted aliphatic heterocyclic ring; a substituted or unsubstituted aromatic heterocyclic ring; or a fused ring thereof.

In the present invention, examples of the “aromatic hydrocarbon ring” include, but are not limited to, a phenyl group, a naphthyl group, an anthracenyl group, etc.

In the present invention, the “aliphatic heterocyclic ring” means an aliphatic ring containing at least one heteroatom.

In the present invention, “aromatic heterocyclic ring” means an aromatic ring containing at least one heteroatom.

In the present invention, “substituted” means that a hydrogen atom bonded to a carbon atom of a compound is replaced with another substituent, and the position of substitution is not limited as long as it is a position where a hydrogen atom is substituted, that is, a position where a hydrogen atom may be substituted with a substituent. When two or more hydrogen atoms are substituted, the two or more substituents may be the same as or different from each other. The substituent may be at least one substituent selected from the group consisting of hydrogen, a cyano group, a nitro group, a halogen group, a hydroxy group, an alkyl group having 1 to 30 carbon atoms, an alkenyl group having 2 to 30 carbon atoms, an alkynyl group having 2 to 24 carbon atoms, a heteroalkyl group having 2 to 30 carbon atoms, an aralkyl group having 6 to 30 carbon atoms, an aryl group having 5 to 30 carbon atoms, a heteroaryl group having 2 to 30 carbon atoms, a heteroarylalkyl group having 3 to 30 carbon atoms, an alkoxy group having 1 to 30 carbon atoms, an alkylamino group having 1 to 30 carbon atoms, an arylamino group having 6 to 30 carbon atoms, an aralkylamino group having 6 to 30 carbon atoms, a heteroarylamino group having 2 to 24 carbon atoms, a substituted or unsubstituted alkylsilyl group having 1 to 30 carbon atoms, a substituted or unsubstituted arylsilyl group having 6 to 30 carbon atoms, and a substituted or unsubstituted aryloxy group having 6 to 30 carbon atoms, without being limited to these examples.

In the present invention, “inducing differentiation” includes both inducing differentiation of progenitor cells such as monocytes or hematopoietic stem cells into dendritic cells in a phenotypically and functionally immature stage, and inducing the differentiation of these immature dendritic cells into phenotypically and functionally mature dendritic cells according to their intended use.

In the present invention, “immature dendritic cells” refers to dendritic cells that are found in the early stage of maturation, and, like mature dendritic cells, do not express cell surface markers such as CD14, express HLA-DR, CD86, CD80, CD83 or CD40 at low levels, and express CD1a and CCR1, CCR2, CCR5 and CXCR1 at normal levels. Differentiation of immature dendritic cells is initiated by receiving various signals, and this differentiation leads to complete differentiation or partial differentiation depending on the combination of signals received. Immature dendritic cells are not able to activate T cells even when in contact with the T cells because they express low levels of inflammatory cytokines.

In the present invention, “mature dendritic cells” means cells formed by maturation of immature dendritic cells, and means cells that express cell surface markers involved in B cell and T cell activity, such as MHC class I or MHC class II (HLA-DR), cell adhesion factors (CD54, CD18, and CD11), and co-stimulatory factors (e.g., CD86, CD80, CD83, or CD40), at high levels or relatively increased levels compared to immature dendritic cells. Typically, mature dendritic cells express high levels of CCR7 and CXCR4.

In the present invention, the “subject” may include, without limitation, mammals, including dogs, cats, rats, livestock, humans, etc., birds, reptiles, farmed fish, etc.

### Advantageous Effects

The present invention relates to a composition for inducing differentiation of dendritic cells including a metal-organic framework and the use thereof. The composition has the effect of not only increasing the expression of dendritic cell surface protein markers, but also inducing differentiation into dendritic cells with increased viability and migratory ability when administered to a subject.

### BRIEF DESCRIPTION OF DRAWINGS

FIG. 1 is a front view of the structure of a metal-organic framework according to one embodiment of the present invention.

FIG. 2 is a side view of the structure of a metal-organic framework according to one embodiment of the present invention.

FIG. 3 is a scanning electron microscopy image of a metal-organic framework according to one embodiment of the present invention.

FIG. 4 shows the results of X-ray diffraction analysis of a metal-organic framework according to one embodiment of the present invention.

FIG. 5 shows the results of measuring the N₂ adsorption/desorption isotherm, specific surface area, and pore diameter of a metal-organic framework according to one embodiment of the present invention.

FIG. 6 shows the results of FT-IR analysis of a metal-organic framework according to one embodiment of the present invention.

FIG. 7 shows the results of a cytotoxicity test for a metal-organic framework according to one embodiment of the present invention.

FIG. 8 shows the results of verifying the ability of a metal-organic framework according to one embodiment of the present invention to induce dendritic cell differentiation and the activation of differentiated dendritic cells.

### BEST MODE

The present invention relates to a composition for inducing differentiation of dendritic cells including a metal-organic framework (MOF).

### MODE FOR INVENTION

Hereinafter, embodiments of the present invention will be described in detail so that those skilled in the art to which the present invention pertains can easily carry out the present invention. However, the present invention may be embodied in various different forms and is not limited to the embodiments described herein.

In the present specification, the “metal-organic framework (MOF)” refers to a porous material in which metal clusters and organic linkers (or organic bridging ligands) are connected by coordination bonds to form a three-dimensional structure. Various MOFs may be created depending on the choice of the metal ion and the organic ligand. The MOF is characterized by a porous property in which pores exist in the structure, and the pore size, porosity, three-dimensional structure, surface area, etc. of the MOF may be designed in various ways depending on the types of metal ion and organic ligand constituting the MOF and a method of bonding between them. Due to this porous property, the MOFs not only have a very large surface area, but also have an open pore structure, allowing the movement of a large amount of molecules or solvents therethrough compared to other porous materials known to date. When the MOFs are used as catalysts or gas storage materials, they may advantageously maximize efficiency due to their numerous active sites. In addition, the MOFs are not easily deformed at high temperatures and have a rigid skeleton, so they have excellent chemical and thermal stability.

Accordingly, the metal-organic framework (MOF) is known to perform various functions such as gas storage, catalyst, drug delivery, and chemical sensor, as it has the property of transporting large quantities of molecules or solvents and storing catalysts or gases by utilizing its porous structural characteristics.

Meanwhile, as described above, “dendritic cells” are the most potent antigen-presenting cells (professional antigen-presenting cells; APCs) that play a crucial role in inducing immunity and regulating immunity. In particular, they are composed of a heterogeneous population with a phenotype distinct from that of macrophages, and are characterized by being potent antigen-presenting cells, unlike B cells and macrophages, which have relatively weak antigen-presenting capabilities.

Since dendritic cells usually account for only about 1% of human peripheral blood mononuclear cells (PBMCs), methods for differentiating progenitor cells into dendritic cells have been studied to obtain a number of cells that are clinically applicable.

Specifically, the process of differentiation of dendritic cells includes ex vivo culture of peripheral blood-derived monocytes or hematopoietic stem cells from peripheral blood, umbilical cord blood, or bone marrow using cytokines, and is divided into two major stages. In the first step, differentiation from progenitor cells such as monocytes and hematopoietic stem cells into dendritic cells that are phenotypically and functionally immature is induced, and the second step is a step of differentiating these immature dendritic cells into dendritic cells using a differentiation inducer capable of inducing phenotypic and functional maturation according to their intended use.

In particular, the characteristics and functions of dendritic cells differentiated in the second step are clearly distinguished as immune enhancement or immune suppression depending on the characteristics of the differentiation inducer. Therefore, it is necessary to appropriately regulate the differentiation of dendritic cells in vitro to provide optimal conditions suitable for the symptoms of various immune diseases.

Specifically, cytokine cocktails, which are combinations of various cytokines, pathogen-associated molecular patterns (PAMPs) commonly present in infectious agents or toll-like receptor ligands for these PAMPs, detoxified and sterilized bacteria, and necrosis-induced cancer cells, have been developed and utilized as dendritic cell differentiation inducers. However, the differentiation inducers showed an increase in immunological activity and function in in vitro analysis, but did not show a sufficient level of increase in immunological activity in clinical studies on humans. This is believed to be because dendritic cells produced ex vivo have very low production of IL-12, which is essential for inducing cell-mediated immunity, have low viability and migratory ability when administered in vivo, and lose function of dendritic cells due to a systemic immunosuppressive environment.

Accordingly, the present invention is intended to provide a composition for inducing differentiation and maturation of dendritic cells, which can overcome the above-mentioned problems and improve the immune activity, viability, and migratory ability of dendritic cells.

Specifically, in the present invention, it has been found that the use of a metal-organic framework (MOF) as a differentiation inducer capable of differentiating immature dendritic cells into mature dendritic cells can overcome the above-described problems in the differentiation process, thereby not only increasing the expression of surface protein markers of differentiated dendritic cells, but also inducing dendritic cells with increased viability and migratory ability when administered into a subject. Based on this finding, the metal-organic framework has been specified.

A composition for inducing differentiation of dendritic cells according to one embodiment of the present invention includes a metal-organic framework (MOF).

The metal-organic framework includes: a metal cluster; and a ligand compound represented by Formula 1 below that coordinates to the metal cluster:

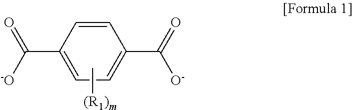

wherein
m is an integer ranging from 0 to 4, and
R₁ is selected from the group consisting of hydrogen, deuterium, a cyano group, a nitro group, an amino group, a halogen group, a hydroxy group, a substituted or unsubstituted alkylthio group having 1 to 4 carbon atoms, a substituted or unsubstituted alkyl group having 1 to 30 carbon atoms, a substituted or unsubstituted cycloalkyl group having 3 to 20 carbon atoms, a substituted or unsubstituted alkenyl group having 2 to 30 carbon atoms, a substituted or unsubstituted alkynyl group having 2 to 24 carbon atoms, a substituted or unsubstituted aralkyl group having 7 to 30 carbon atoms, a substituted or unsubstituted aryl group having 6 to 30 carbon atoms, a substituted or unsubstituted heteroaryl group having 1 to 60 carbon atoms, a substituted or unsubstituted heteroarylalkyl group having 2 to 30 carbon atoms, a substituted or unsubstituted alkoxy group having 1 to 30 carbon atoms, a substituted or unsubstituted alkylamino group having 1 to 30 carbon atoms, a substituted or unsubstituted arylamino group having 6 to 30 carbon atoms, a substituted or unsubstituted aralkylamino group having 7 to 30 carbon atoms, a substituted or unsubstituted heteroarylamino group having 1 to 24 carbon atoms, a substituted or unsubstituted alkylsilyl group having 1 to 30 carbon atoms, a substituted or unsubstituted arylsilyl group having 6 to 30 carbon atoms, and a substituted or unsubstituted aryloxy group having 6 to 30 carbon atoms.

Meanwhile, R₁ is selected from the group consisting of an amino group, a substituted or unsubstituted alkylamino group having 1 to 30 carbon atoms, a substituted or unsubstituted arylamino group having 6 to 30 carbon atoms, a substituted or unsubstituted aralkylamino group having 7 to 30 carbon atoms, and a substituted or unsubstituted heteroarylamino group having 1 to 24 carbon atoms.

Specifically, the ligand may be a compound wherein R₁ is an amino group. More specifically, it may be a compound represented by Formula 2 below:

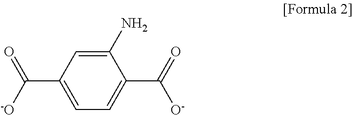

The —COO— group of the ligand compound represented by Formula 2 may form a coordination bond with the metal cluster to form the metal-organic framework.

As described above, the metal-organic framework of the present invention is able to induce differentiation of immature dendritic cells into mature dendritic cells, and thus has the effect of increasing the expression levels of surface protein markers of the differentiated dendritic cells. Thereby, the immune-inducing activity of dendritic cells may be maximized.

The metal-organic framework includes at least one metal or metal ion selected from the group consisting of aluminum (Al), iron (Fe), and zirconium (Zr).

Specifically, the metal-organic framework may be synthesized using the selected metal or metal ion, and specifically, it may include the metal or metal ion and an organic ligand coordinated thereto. Specifically, the metal portion of the metal-organic framework may be composed of at least one metal or metal ion selected from the group consisting of aluminum (Al, Al³⁺), iron (Fe, Fe³⁺), and zirconium (Zr, Zr⁴⁺).

In one embodiment, the metal-organic framework may be an aluminum-based MOF, an iron-based MOF, and/or a zirconium-based MOF.

In one embodiment, the aluminum-based MOF may include, but is not limited to, at least one selected from the group consisting of Al-MIL-53, Al-MIL-53-NH₂, Al-MIL-88, Al-MIL-88-NH₂, Al-MIL-100, Al-MIL-100-NH₂, Al-MIL-101, Al-MIL-101-NH₂, Al-MIL-125, and Al-MIL-125-NH₂.

In one embodiment, the iron-based MOF may include, but is not limited to, at least one selected from the group consisting of Fe-MIL-53, Fe-MIL-53-NH₂, Fe-MIL-88, Fe-MIL-88-NH₂, Fe-MIL-100, Fe-MIL-100-NH₂, Fe-MIL-101, Fe-MIL-101-NH₂Fe-MIL-125, and Fe-MIL-125-NH₂.

In one embodiment, the zirconium-based MOF may include, but is not limited to, at least one selected from the group consisting of UIO-66, UIO-66-NH₂, UIO-67, UIO-67-NH₂, PCN-128, PCN-222, PCN-223, PCN-224, MOF-525, MOF-545, MOF-801, MOF-808, and MOF-867. Specifically, according to one example, it was confirmed that UIO-66-NH₂, a representative example of the zirconium-based MOF, could effectively induce differentiation of immature dendritic cells into mature dendritic cells.

A pharmaceutical composition according to another embodiment of the present invention may include the above-described composition.

The pharmaceutical composition may include a pharmaceutically acceptable carrier. The term “pharmaceutically acceptable carrier” may refer to a carrier or diluent that does not inhibit the biological activity and properties of the compound to be injected without stimulating a living organism. Here, “pharmaceutically acceptable” means that the activity of the active ingredient is not inhibited and there is no more than adaptable toxicity in the subject of application (prescription). Any type of pharmaceutically acceptable carrier that is commonly used in the art may be used in the pharmaceutical composition. Non-limiting examples of the carrier include lactose, dextrose, maltodextrin, sucrose, sorbitol, mannitol, xylitol, erythritol, maltitol, glycerol, ethanol, starch, gum acacia, alginate, gelatin, calcium phosphate, calcium silicate, cellulose, methyl cellulose, microcrystalline cellulose, polyvinyl pyrrolidone, water, saline solution, sterile water, Ringer's solution, buffered saline, albumin injection solution, methylhydroxybenzoate, propylhydroxybenzoate, talc, magnesium stearate, or mineral oil. These may be used alone or in combination of two or more. The pharmaceutical composition including a pharmaceutically acceptable carrier in addition to the active ingredient may be prepared as an oral formulation or a parenteral formulation according to the route of administration by a conventional method known in the art. The pharmaceutical composition may be formulated and used in the form of oral formulations such as powders, granules, tablets, capsules, suspensions, emulsions, syrups, or aerosols, external preparations, suppositories, or sterile injectable solutions according to a conventional method.

When the pharmaceutical composition is formulated, it may be prepared using diluents or excipients such as commonly used fillers, extenders, binders, wetting agents, disintegrants, or surfactants, without being limited thereto.

When the pharmaceutical composition is prepared as oral formulations, it may be prepared as formulations such as powders, granules, tablets, pills, dragees, capsules, liquids, gels, syrups, suspensions, wafers, etc., using suitable carriers according to methods known in the art. In this case, examples of suitable pharmaceutically acceptable carriers include sugars such as lactose, glucose, sucrose, dextrose, sorbitol, mannitol, xylitol, etc.; starches such as corn starch, potato starch, and wheat starch; celluloses such as cellulose, methylcellulose, ethylcellulose, sodium carboxymethylcellulose, and hydroxypropylmethylcellulose; polyvinyl pyrrolidone, water, methyl hydroxybenzoate, propyl hydroxybenzoate, magnesium stearate, mineral oil, malt, gelatin, talc, polyols, vegetable oils, and the like. For formulation, the composition may be formulated using diluents and/or excipients such as fillers, extenders, binders, wetting agents, disintegrants, and surfactants, as needed.

When the pharmaceutical composition is prepared as parenteral formulations, it may be formulated in the form of an injection, a transdermal drug delivery system, a nasal spray, and a suppository using suitable carriers according to methods known in the art. For formulation as an injection, suitable carriers include sterile water, ethanol, polyols such as glycerol or propylene glycol, or mixtures thereof, and preferably, Ringer's solution, phosphate-buffered saline (PBS) containing triethanolamine, sterile water for injection, or an isotonic solution such as 5% dextrose may be used. For formulation as a transdermal drug delivery system, the composition may be formulated in the form of ointments, creams, lotions, gels, topical solutions, pastes, liniments, aerosols, etc. For nasal spray, the composition may be formulated in the form of an aerosol spray using a suitable propellant such as dichlorofluoromethane, trichlorofluoromethane, dichlorotetrafluoroethane, or carbon dioxide. For formulation as a suppository, witepsol, Tween 61, polyethylene glycol, cocoa butter, laurin fat, polyoxyethylene sorbitan fatty acid ester, polyoxyethylene stearate, or sorbitan fatty acid ester may be used as a base.

The pharmaceutical composition may be administered in a pharmaceutically effective amount. The term “pharmaceutically effective amount” refers to an amount sufficient to treat or prevent a disease at a reasonable benefit/risk ratio applicable to any medical treatment or prevention. The effective dose level of the pharmaceutical composition may be determined depending on factors including the severity of the disease, the activity of the drug, the patient's age, weight, health, and sex, the patient's sensitivity to the drug, the time of administration of the composition of the present invention used, the route of administration, the excretion rate, the duration of treatment, drugs used in combination or concurrently with the composition of the present invention used, as well as other factors well known in the medical field. The pharmaceutical composition of the present invention may be administered alone or in combination with an ingredient known to exhibit a therapeutic effect on inflammation, allergic diseases, atopic dermatitis, or asthma. It is important to administer the pharmaceutical composition in the minimum amount that can exhibit the maximum effect without causing side effects, in view of all the above-described factors.

The dose of the pharmaceutical composition may be determined by a person skilled in the art in consideration of the intended use, the severity of the disease, the patient's age, weight, sex, and medical history, or the type of substance used as the active ingredient. For example, the pharmaceutical composition of the present invention may be administered at a dose of about 0.1 ng to about 1,000 mg/kg, preferably 1 ng to about 100 mg/kg, per adult, and the composition of the present invention may be administered once a day or administered in divided doses several times, but the frequency of administration of the composition is not particularly limited thereto. The dose or frequency of administration does not limit the scope of the present invention in any way.

Meanwhile, one embodiment of the present invention provides a method for inducing differentiation of dendritic cells in a subject, including a step of administering the composition for inducing differentiation of dendritic cells to the subject. The same contents as described above may be equally applied to the method.

A method for inducing differentiation of dendritic cells according to another embodiment of the present invention includes a step of treating immature dendritic cells with the composition, followed by culturing.

The immature dendritic cells are cells obtained by treating bone marrow cells with granulocyte-macrophage colony-stimulating factor (GM-CSF).

However, the immature dendritic cells are not limited to cells obtained by the above method, and the type thereof is not limited as long as it is capable of differentiating into mature dendritic cells.

Meanwhile, the bone marrow cells may be bone marrow-derived hematopoietic stem cells, without being limited thereto.

According to another embodiment of the present invention, a dendritic cell is a cell differentiated by the method for inducing differentiation.

The dendritic cell has increased expression of at least one surface protein marker selected from the group consisting of CD40, CD80, CD86, MHC class I, and MHC class II.

Specifically, dendritic cells induced to differentiate by treatment with the composition for inducing differentiation of dendritic cells according to the present invention are characterized by having increased expression levels of surface protein markers such as MHC-I, MHC-II, CD40, CD80, CD83, and CD86, and thus are characterized by being able to induce immune activation such as inducing strong T cell proliferation and promoting the production of IFN-γ and IL-2 from T cells.

Hereinafter, the present invention will be described in more detail through examples. These examples are only intended to illustrate the present invention in more detail, and it will be apparent to those skilled in the art that the scope of the present invention according to the subject matter of the present invention is not limited by these examples.

### PRODUCTION EXAMPLE

### Synthesis of UIO-66-NH₂

100 ml of N,N-dimethylformamide (DMF) and 3.8 g of benzoic acid were added to a 100-ml container and stirred. After the benzoic acid was completely dissolved, 120 mg of ZrCl₄ was additionally added to the container, followed by stirring. Next, when the ZrCl₄ was completely dissolved, 2-aminoterephthalic acid was additionally added to the container, followed by stirring to dissolve all of the reagents. Next, the container was sealed with Teflon tape, followed by reaction at 120° C. for 24 hours.

Thereafter, the particles synthesized through the reaction were washed three times with each of DMF and EtOH, and then dried at 80° C. to obtain powder.

### Experimental Example

### Analysis of Synthesis Results

For measurement using a scanning electron microscope (SEM), 2 mg to 3 mg of a powder sample were placed in an Eppendorf tube and 100 μL of ethanol was added thereto, followed by bath sonication. Thereafter, 10 μL of the solution was dropped on a silicon wafer and dried in an oven at 80° C. The silicon wafer with the dried sample was attached to a SEM mount using carbon tape, and the sample was imaged in a SEM instrument (Zeiss, Model ULTRA PLUS).

Meanwhile, for X-ray diffraction (XRD) measurement, a powder sample was carefully placed on the XRD sample holder, and the sample was compressed by pressing the same with an appropriate force using slide glass. Thereafter, all powder scattered around the sample holder was carefully removed, and then the sample holder was transferred to an X-ray diffractometer (Bruker, model D2 Phaser) and set, and then measurements were performed in the range of 2° C. to 30° C.

Meanwhile, for the measurement of the N₂ adsorption/desorption isotherm, BET surface area and pore diameter, about 40 mg of a completely dried sample was prepared and carefully placed in a glass sampler using a glass funnel, and all powder accumulated around the glass tube outside the sample container at the bottom was removed. Thereafter, an upper filter cap was attached and degassing was performed under vacuum by heat treatment at 120° C. for 12 hours in a pretreatment instrument. The pretreated sample was weighed using a microbalance, and the correct weight of the sample was calculated by subtracting the premeasured weight of the empty cell. The glass sampler was placed on an N₂ adsorption-desorption instrument (MICROTRAC, model BELSORP MINI X), and measurements were performed.

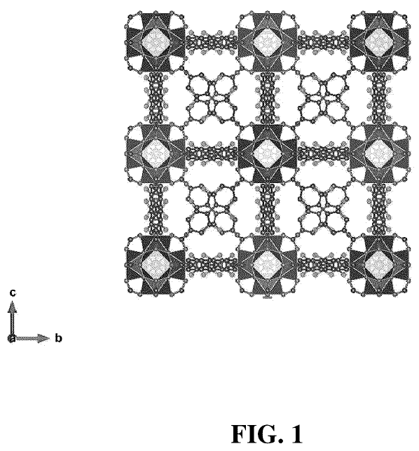

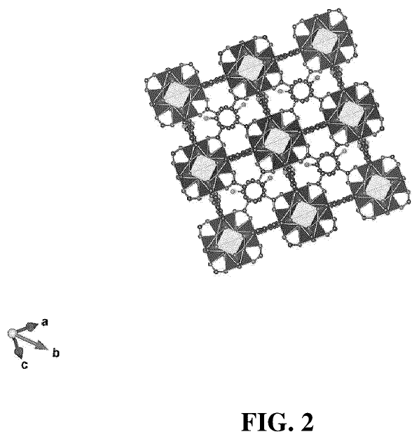

FIGS. 1 and 2 show the structural images of the UIO-66-NH₂ particles.

FIG. 1 is a front view of the structure of UIO-66-NH₂, and FIG. 2 is a side view of the structure of UIO-66-NH₂.

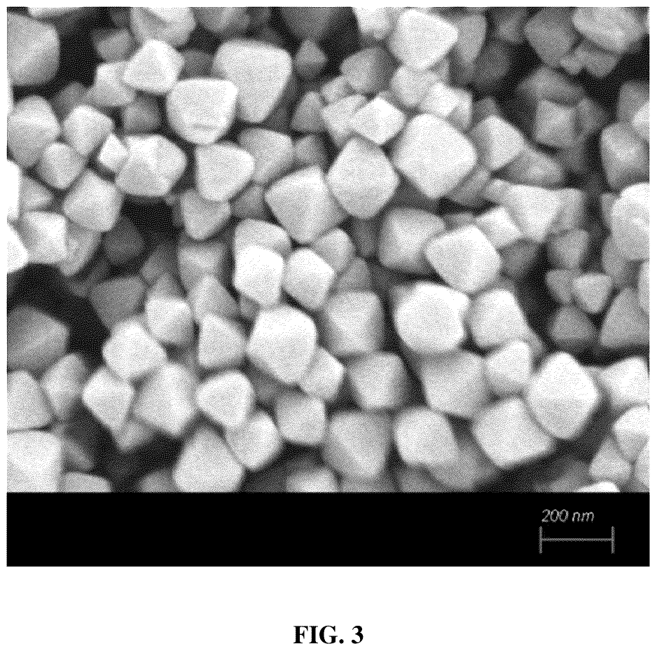

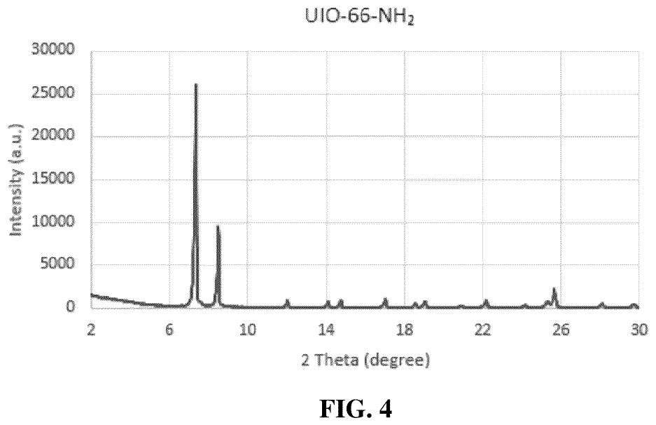

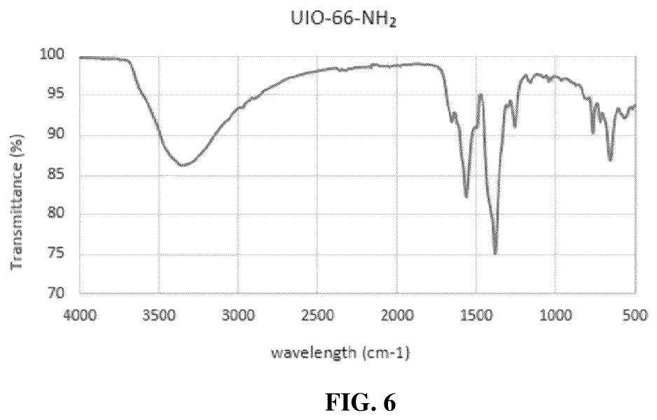

FIG. 3 shows the results of scanning electron microscope observation of UIO-66-NH₂, FIG. 4 shows the results of X-ray diffraction analysis, and FIG. 6 shows the results of FT-IR measurement. Referring thereto, it can be confirmed that the produced particles were synthesized with the crystal structure of UIO-66-NH₂.

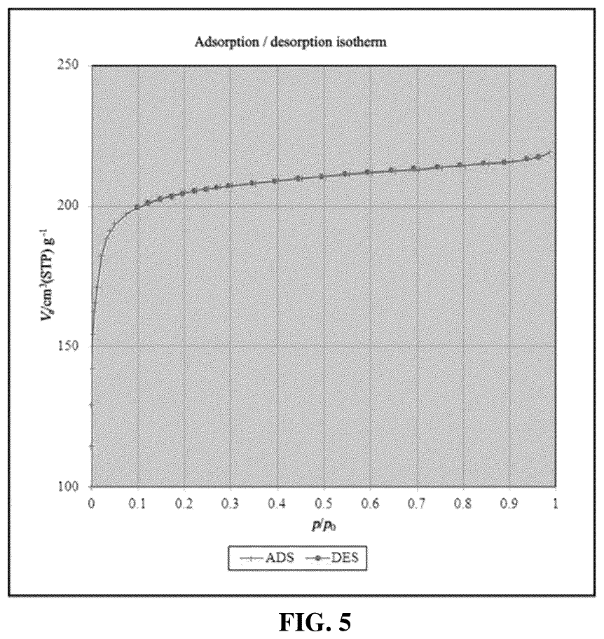

Meanwhile, FIG. 5 shows the results of measurement of the N₂ adsorption/desorption isotherm, specific surface area, and pore diameter. The results of BET (Brunauer-Emmett-Teller) analysis through N₂ adsorption showed that the specific surface area of UIO-66-NH₂ particles was 792.02 m²/g and the pore diameter thereof was 1.71 nm.

### Cytotoxicity Evaluation

Mouse fibroblasts (L929) were seeded in a 96-well plate at a density of 9,000 cells per well and incubated at 37° C. for 24 hours. Then, the existing medium in each well was removed, and UIO-66-NH₂ was suspended in media at concentrations of 100 μg/mL, 50 μg/mL, 10 μg/mL, 5 μg/mL, and 1 μg/mL, respectively, thus preparing MOF suspensions. 100 μL of each suspension with each UIO-66-NH₂ concentration was added to each well. Next, each suspension was incubated at 37° C. for 24 hours and washed three times with PBS to remove the medium and the particles attached to the plate and cell surface. Next, 10% Ez-Cytox was added to each well which was then incubated at 37° C. for 1 hour. Thereafter, the absorbance was measured at 450 nm using a microplate reader (reference wavelength: 600 nm to 650 nm).

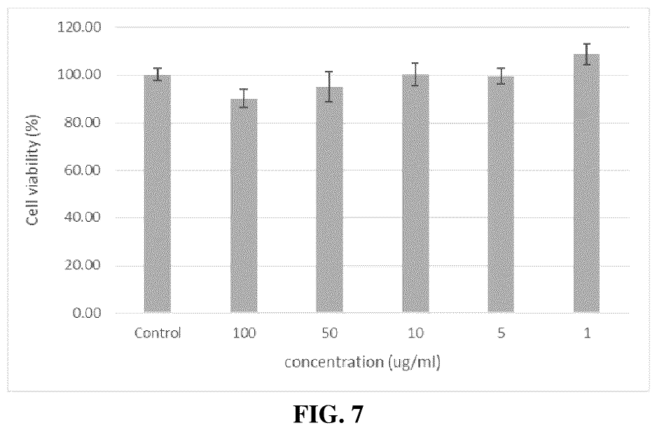

FIG. 7 shows the results of evaluating cytotoxicity using the above-described method. Referring to FIG. 7, it was confirmed that there was no cytotoxicity in the MOF suspension with a concentration of up to 100 μg/mL.

Verification of the Ability to Induce Differentiation into Dendritic Cells and the Activation of Differentiated Dendritic Cells

The ability of the MOF treatment to induce differentiation of immature dendritic cells into mature dendritic cells of the present invention and the immune activation of differentiated dendritic cells were verified using the following methods.

GM-CSF/recombinant mouse granulocyte-macrophage colony-stimulating factor (GM-CSF) was purchased from Biolegend (CA, USA), and RPMI 1640 medium (Gibco, Cat No. 22400), fetal bovine serum (FBS; Gibco, Cat No. 16000044), and penicillin/streptomycin (Gibco, Cat No. 15140163) were purchased from Thermo Fisher Scientific (MA, USA).

Specifically, bone marrow cells were treated with granulocyte-macrophage colony-stimulating factor (GM-CSF) (Biolegend (CA, USA)) at a concentration of 20 ng/ml for 6 days to obtain immature bone marrow dendritic cells differentiated from the bone marrow cells. Thereafter, the immature bone marrow dendritic cells were seeded in a 96-well plate at a density of 1×10⁵ cells/well and cultured in RPMI medium (RPMI 1640, (Gibco, Cat No. 22400)). Thereafter, suspensions of UIO-66-NH₂ prepared at concentrations of 25, 50, and 100 μg/mL were added to the immature bone marrow dendritic cells which were then co-cultured at 37° C. for 24 hours.

Meanwhile, a group treated with 50 ng/mL lipopolysaccharide (LPS) was prepared as a positive control group so that it could be compared with the test group.

To analyze maturation markers of the mature bone marrow dendritic cells differentiated from the immature dendritic cells through 24-hour culture, cells were harvested after incubation, labeled with fluorescently conjugated primary antibodies, and then analyzed using a CytoFLEX flow cytometer (Beckman Coulter, CA, USA).

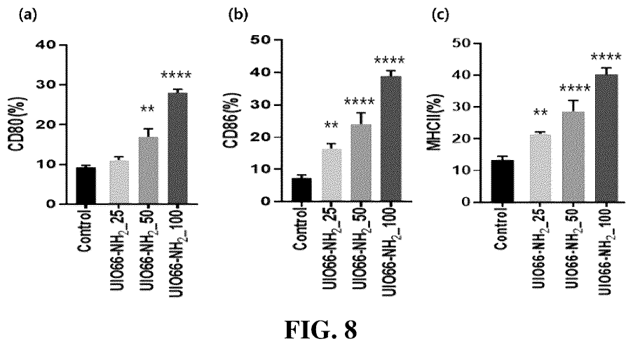

FIG. 8 shows the results of verifying the ability to induce differentiation and the activation of differentiated dendritic cells using the above-described method. Referring to FIG. 8, it was confirmed that immature dendritic cells differentiated into mature dendritic cells when treated with the MOF suspension, and that, in the case of differentiated dendritic cells, the expression of surface protein markers increased in a manner dependent on the concentration of the MOF suspension used for treatment.

Although the preferred embodiments of the present invention have been described in detail above, the scope of rights of the present invention is not limited thereto, and various modifications and improvements can be made by those skilled in the art without departing from the basic concept of the present invention as defined in the following claims and also fall within the scope of rights of the present invention.

This project (result) is the result of a local government-university cooperation-based regional innovation project (2021RIS-001) supported by the National Research Foundation of Korea funded by the Ministry of Education in 2024.
[Project Serial Number] 1345370811
[Project Number] 2021RIS-001
[Government Department] The Ministry of Education (P13)
[Project Management Agency] The National Research Foundation of Korea
[Research Project Name] Local government-university cooperation-based regional innovation project
[Research Task Name] Local government-university cooperation-based regional innovation project (Chungbuk regional innovation platform)
[Contribution Rate]
[Agency Carrying Out Project] (Chungbuk regional innovation platform) Chungbuk National University
[Research Period] Mar. 1, 2024 to Feb. 28, 2025

### INDUSTRIAL APPLICABILITY

The present invention relates to a composition for inducing differentiation of dendritic cells including a metal-organic framework and the use thereof.

1. A composition for inducing differentiation of dendritic cells comprising a metal-organic framework (MOF).

2. The composition of claim 1, wherein the metal-organic framework comprises:
a metal cluster; and
a ligand compound represented by Formula 1 below that coordinates to the metal cluster: 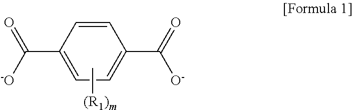
wherein
m is an integer ranging from 0 to 4, and
R₁ is selected from the group consisting of hydrogen, deuterium, a cyano group, a nitro group, an amino group, a halogen group, a hydroxy group, a substituted or unsubstituted alkylthio group having 1 to 4 carbon atoms, a substituted or unsubstituted alkyl group having 1 to 30 carbon atoms, a substituted or unsubstituted cycloalkyl group having 3 to 20 carbon atoms, a substituted or unsubstituted alkenyl group having 2 to 30 carbon atoms, a substituted or unsubstituted alkynyl group having 2 to 24 carbon atoms, a substituted or unsubstituted aralkyl group having 7 to 30 carbon atoms, a substituted or unsubstituted aryl group having 6 to 30 carbon atoms, a substituted or unsubstituted heteroaryl group having 1 to 60 carbon atoms, a substituted or unsubstituted heteroarylalkyl group having 2 to 30 carbon atoms, a substituted or unsubstituted alkoxy group having 1 to 30 carbon atoms, a substituted or unsubstituted alkylamino group having 1 to 30 carbon atoms, a substituted or unsubstituted arylamino group having 6 to 30 carbon atoms, a substituted or unsubstituted aralkylamino group having 7 to 30 carbon atoms, a substituted or unsubstituted heteroarylamino group having 1 to 24 carbon atoms, a substituted or unsubstituted alkylsilyl group having 1 to 30 carbon atoms, a substituted or unsubstituted arylsilyl group having 6 to 30 carbon atoms, and a substituted or unsubstituted aryloxy group having 6 to 30 carbon atoms.

3. The composition of claim 2, wherein R₁ is selected from the group consisting of an amino group, a substituted or unsubstituted alkylamino group having 1 to 30 carbon atoms, a substituted or unsubstituted arylamino group having 6 to 30 carbon atoms, a substituted or unsubstituted aralkylamino group having 7 to 30 carbon atoms, and a substituted or unsubstituted heteroarylamino group having 1 to 24 carbon atoms.

4. The composition of claim 1, wherein the metal-organic framework comprises at least one metal or metal ion selected from the group consisting of aluminum (Al), iron (Fe), and zirconium (Zr).

5. A method for inducing differentiation of dendritic cells comprising a step of treating immature dendritic cells with the composition according to claim 1, followed by culturing.

6. The method of claim 5, wherein the immature dendritic cells are cells obtained by treating bone marrow cells with granulocyte-macrophage colony-stimulating factor (GM-CSF).

7. A dendritic cell differentiated by the method for inducing differentiation according to claim 5.

8. The dendritic cell of claim 7, wherein the dendritic cell has increased expression of at least one surface protein marker selected from the group consisting of CD40, CD80, CD86, MHC class I, and MHC class II.
